(19) United States
(12) Patent Application Publication
(10) Pub. No.: US 2017/0152294 A1
(43) Pub. Date: Jun. 1, 2017

(54) METHOD FOR EXTRACTING SOLUBLE PROTEINS FROM MICROALGAL BIOMASS

(71) Applicant: ROQUETTE FRERES, Lestrem (FR)

(72) Inventor: Samuel PATINIER, Quesnoy sur Deule (FR)

(21) Appl. No.: 15/322,199

(22) PCT Filed: Jul. 16, 2015
(86) PCT No.: PCT/FR2015/051940

§ 371 Date: Dec. 27, 2016

(30) Foreign Application Priority Data
Jul. 18, 2014 (FR) 14 56944

(51) Int. Cl. C07K 14/405; C07K 1/36

(52) U.S. Cl. CPC C07K 14/405; C07K 1/36

(57) ABSTRACT

Method for preparing a protein isolate of the biomass of microalgae of the genus Chlorella, includes: supplying a microalgal biomass produced by fermentation; optionally washing the biomass so as to eliminate soluble interstitial compounds, thermal permeabilization of the biomass at a temperature between 50 and 150° C., for between 10 seconds and 5 minutes; eliminating the thus permeabilized biomass by a solid-liquid separation technique selected from the group consisting of frontal or tangential filtration, flocculation and centrifuging, more specifically multi-stage centrifuging, in order to obtain a soluble fraction; optionally recovering and clarifying the soluble fraction thus obtained by microfiltration in such a way as to remove residual insoluble elements therefrom; ultrafiltration of the soluble fraction on a membrane with a cut-off threshold lower than 5 kDa, preferably between 1 and 5 kDa, in order to produce a soluble protein isolate; then evaporation, pasteurization and atomization of the protein isolate.

The present invention relates to a method for extracting soluble proteins from microalgal biomass.

The present invention also relates to the microalgal protein isolates obtained in this way.

### PRESENTATION OF THE PRIOR ART

It is well known to those skilled in the art that chlorellae are a potential source of food, since they are rich in proteins and other essential nutrients.

They are described as containing 45% of proteins, 20% of fats, 20% of carbohydrates, 5% of fibers and 10% of minerals and vitamins.

Given their abundance and their amino acid profile, microalgal proteins are thus considered as an alternative source to soy or pea proteins in food.

The protein fraction may also be exploited as a functional agent in the cosmetic, or even pharmaceutical, industries.

However, developments in food applications for microalgal proteins have not been significant, since the presence in said fractions of undesirable compounds (such as chlorophyll) leads to undesired changes in color, flavor and structure of the food compositions containing them.

To increase their potential in food applications and also to increase their commercial value, these proteins must therefore be extracted from the microalgae without affecting their molecular structure.

“Soft” extraction techniques would therefore be necessary to isolate proteins with high solubilities and good technical and functional properties, but the rigidity of microalgal cell walls, especially of green microalgae, is fundamentally in contradiction to this, since it disrupts the extraction and integrity of the intracellular proteins.

Thus, on the contrary, conventionally “hard” physical or chemical conditions are employed to break the microalgal cell wall.

Numerous studies thus propose technologies of alkaline dissolution type, extraction by organic solvent type or high-pressure homogenization type.

In these technological choices, the denaturing of proteins was not however considered to be bothersome, since most of these methods were developed for purposes of analyses or intended to provide a substrate for the enzymatic digestion producing protein hydrolyzates.

However, an effective disintegration method preserving the integrity of the cell components has a duty to maximize not only the yield, but also the quality of the products extracted.

In other words, a method for optimized disintegration of the wall must for example avoid:
chemical contamination of the targeted products,
using a breaking energy which is too high; the latter possibly causing irreversible denaturation or degradation of the intracellular molecules of interest.

Moreover, for large-scale productions, it is important for the method chosen to be transposable to this scale.

Finally, the introduction of this cell disintegration step must be easy and must not have a negative impact on the subsequent method/treatment steps.

All these limitations influence the efficiency of the disintegration method and by the same token its energy consumption.

This is why the bead mill technology is preferred, since it is considered to be efficient for releasing intracellular proteins in their native form.

In a bead mill, the cells are agitated in suspension with small spherical particles. The breaking of the cells is caused by the shear forces, the milling between the beads, and the collisions with beads.

The description of an appropriate bead mill is, for example, given in the U.S. Pat. No. 5,330,913. These beads break the cells so as to release the cell content therefrom. A suspension of particles of smaller size than the cells of origin is then obtained in the form of an “oil-in-water” emulsion.

This emulsion is generally atomized and the water is eliminated, leaving a dry powder containing, however, a heterogeneous mixture composed of cell debris, interstitial soluble compounds, and oil.

The difficulty to be solved in the use of these cell disintegration technologies is the isolation of solely the intracellular content (to the exclusion of the membrane debris, sugars, fibers and fats) and the preservation, especially, of the quality of the protein load.

In the case of the microalga of the genus Tetraselmis sp, Anja Schwenzfeier et al (Bioresource Technology, 2011, 102, 9121-9127) proposed a method guaranteeing the solubility and the quality of the aminogram of the proteins isolated and with contaminants (such as coloring substances) removed, comprising the following steps:
cell disintegration by bead mill,
centrifugation of the milled microalgal suspension,
dialysis of the supernatant,
passage over ion-exchange resin,
dialysis of the eluate,
discoloration, then
washing and resuspending.

However, this laboratory method (for treating 24 g of biomass) cannot be scaled up to an industrial scale, where the bead mill method is rather used to recover a complete biomass.

Alternative solutions have been proposed, completely changing the technology for releasing the intracellular content of the microalgae, such as pulsed-field electrical treatment.

This is because exposure of biological cells to a high-intensity pulsed electric field can modify the structure of the cell membrane.

The external field causes charging of the membrane. At a sufficient transmembrane voltage (0.5-1 V), the molecular arrangement of the phospholipids changes, which results in the membrane losing its barrier role, making it permeable. Depending on the conditions used, this membrane permeabilization can be reversible or irreversible.

For efficient extraction of the intracellular compounds, those skilled in the art remain, however, advised to bring about an irreversible permeabilization of the membrane, thereby resulting in its disintegration.

This rupture of the membrane then facilitates the release of the cell content and, in the case of the use of a supplementary solvent-extraction technique, also facilitates the penetration of the solvent into the cell.

This technique, although promising, can unfortunately not be extrapolated to an industrial scale for treating a biomass produced in a reactor of 1 to 200 m³.

As a result, there remains an unmet need to provide a technology for weakening microalgal cell walls that is capable of releasing the intracellular content without disintegrating the cell or impairing the quality of the components thereof.

The Applicant company has found that this need can be met by combining a method for the thermal permeabilization of the microalgal cells with steps of centrifugation and membrane separation (microfiltration, ultrafiltration).

The Applicant company thus goes against a technical prejudice which says that thermal methods for cell disruption, just like the shear forces caused by mechanical disintegration, are technologies that are instead used for degrading or denaturing the products originating from microalgae (Richmond, 1986, Handbook of Microalgal Mass Culture. CRC Press, Inc—Molina Grima et al., 2003, Recovery of microalgal biomass and metabolites: process options and economics, Biotechnol. Adv. 20:491-515).

Moreover, once released from the intracellular compartment, the recovery of the molecules can be carried out easily by centrifugation and membrane separation, given that the thermal treatment developed by the Applicant company does not result in the disintegration of the cell wall.

The present invention relates to a method for thermal permeabilization of the biomass of microalgae of the Chlorella genus in such a way as to recover therefrom the soluble fractions which are especially enriched with peptides and polypeptides and with oligosaccharides.

More specifically, the method according to the invention is a method for preparing a protein isolate from the biomass of microalgae of the Chlorella genus, comprising the following steps:
providing a microalgal biomass produced by fermentation,
optionally, washing the biomass so as to eliminate the interstitial soluble compounds,
thermal permeabilization of the biomass at a temperature of between 50 and 150° C., preferably 100 and 150° C., for a duration of between 10 seconds and 5 minutes, preferably for a duration of between 10 seconds and 1 minute,
elimination of the biomass permeabilized in this way by a technique of solid-liquid separation chosen from the group consisting of frontal or tangential filtration, flocculation and centrifugation, more particularly multistage centrifugation, to obtain a soluble fraction,
optionally, recovery and clarification of the soluble fraction obtained in this way by microfiltration so as to remove residual insoluble substances therefrom,
ultrafiltration of the soluble fraction (clarified or unclarified, depending on whether the preceding step of recovery and clarification is or is not carried out, respectively) on a membrane with a cut-off threshold of less than 5 kDa, preferably of between 1 and 5 kDa, so as to obtain a soluble protein isolate, then
evaporation, pasteurization and atomization of said protein isolate.

Choice of the Microalga

Preferably, the microalgae of the Chlorella genus are chosen from the group consisting of Chlorella vulgaris, Chlorella sorokiniana and Chlorella protothecoides, and are more particularly Chlorella protothecoides.

In one particular embodiment, the strain is Chlorella protothecoides (strain UTEX 250—The Culture Collection of Algae at the University of Texas at Austin—USA).

In another particular embodiment, the strain is Chlorella sorokiniana (strain UTEX 1663—The Culture Collection of Algae at the University of Texas at Austin—USA).

The culturing under heterotrophic conditions and in the absence of light conventionally results in the production of a chlorella biomass having a protein content (evaluated by measuring the nitrogen content N×6.25) of 45% to 70% by weight of dry cells.

As will be exemplified hereinafter, this culturing is carried out in two steps:
preculturing in a medium containing glucose and yeast extract for 72 h at 28° C. with agitation, then
culturing for production of the biomass per se in glucose and yeast extract for more than 36 h at 28° C., with agitation and at pH 6.5 adjusted with aqueous ammonia,

which results in approximately 80 g/l of biomass with a protein content (evaluated by N×6.25) of the order of 52% by weight of dry cells.

The biomass is then collected by solid-liquid separation, by frontal or tangential filtration or by any means known, moreover, to those skilled in the art.

Optionally, the Applicant company then recommends washing the biomass in such a way as to eliminate the interstitial soluble compounds by a succession of concentration (by centrifugation)/dilution of the biomass.

For the purposes of the invention, the term “interstitial soluble compounds” is intended to mean all the soluble organic contaminants of the fermentation medium, for example the hydrosoluble compounds such as the salts, the residual glucose, the oligosaccharides with a degree of polymerization (or DP) of 2 or 3, or the peptides.

This biomass purified in this way of its interstitial soluble compounds is then preferentially adjusted to a dry matter of between 15% and 30% by weight, preferably to a dry matter of between 20% and 30%.

Thermal Permeabilization of the Biomass

Thermal treatment is then carried out at a temperature of between 50 and 150° C., preferably 100 and 150° C., for a duration of between 10 seconds and 5 minutes, preferably for a duration of between 10 seconds and 1 minute.

This treatment makes it possible to allow the intracellular components to diffuse into the reaction medium.

Finally, at the end of these steps, the temperature is allowed to cool to a final temperature of between 0° and 10° C., preferably to a temperature of the order of 4° C.

Without wishing to be bound by a particular theory, the Applicant company considers that the thermal treatment, carried out under these operating conditions, could thus act as a membrane weakening process which allows the spontaneous release of the soluble components of the intracellular compartment, or even of the extracellular matrix.

In addition to the ionic substances, organic substances such as carbohydrates (predominantly DP1 and DP2), the peptides and the polypeptides are drained out of the cell.

Conversely, the lipids and hydrophobic organic compounds remain in the cells, thereby clearly demonstrating that the cells are permeabilized and not lyzed or destroyed.

The method according to the invention does not therefore result in the formation of an emulsion, but indeed of an aqueous suspension.

The release of all these soluble substances through the permeabilized membrane is similar to a process of free diffusion of dialysis type.

Consequently, a lag time may be necessary in order to allow sufficient diffusion after the heat treatment which permeabilizes the membrane.

In the literature, the process for pulsed-field permeabilization of yeast membranes in order to extract the proteins therefrom requires from 4 h to 6 h (Ganeva et al., 2003, Analytical Biochemistry, 315, 77-84).

According to the invention, a much shorter reaction time is used, of between 10 seconds and 5 minutes.

The residual biomass is then eliminated by a technique of solid-liquid separation by frontal or tangential filtration, by flocculation, by centrifugation or by any means known, moreover, to those skilled in the art, thereby making it possible to easily recover the soluble fraction from which the microalgal cells have been removed.

Before this step for eliminating the biomass, it is possible to carry out dilution of the permeabilized cells in order to improve the yield and the quality of this solid-liquid separation step.

The resulting soluble fraction is finally essentially composed of proteins (50-80% w/w) and carbohydrates (5-15% w/w).

Membrane Separation

The conventional methods for recovering soluble proteins are generally based on a step of precipitating said proteins with trichloroacetic acid (10% weight/volume) or with ammonium sulfate.

However, these isolations by precipitation follow on from very destructive cell-breaking methods (usually by sonication or homogenization) which, while they make it possible in fact to increase extraction yields, result especially in proteins of low solubility which are denatured.

It is then possible to envision the refunctionalization thereof only by means of their product of hydrolysis (to peptides) by chemical means (lysis with sodium hydroxide), physical means (high-temperature treatment) or enzymatic means (proteolytic enzymes).

The method according to the invention makes it possible, quite to the contrary, to release intact native peptides and polypeptides, all the functionalities of which can still be expressed.

The method of the invention next leads to the isolation of the proteins of interest by membrane fractionation.

The applicant company thus recommends carrying out the method in two steps:
optionally, recovery and clarification of the soluble fraction obtained in this way by microfiltration so as to remove residual insoluble substances therefrom,
ultrafiltration of the soluble fraction (clarified or unclarified, depending on whether the preceding step of recovery and clarification is or is not carried out, respectively) on a membrane with a cut-off threshold of less than 5 kDa, preferably of between 1 and 5 kDa, so as to obtain a soluble protein isolate.

Utilizing these pathways makes it possible to purify the soluble peptides and polypeptides of their residual salts and sugars.

An isolate of soluble proteins is thus obtained, having a protein content of greater than 90% by weight.

The invention will be understood more clearly from the following examples which are intended to be illustrative and nonlimiting.

### EXAMPLES

### Example 1: Production of Chlorella protothecoides by Fed-Batch Fermentation

The strain used is Chlorella protothecoides UTEX 250.

Preculture:
500 ml of medium in a 2 l conical flask;
Composition of the medium (in g/l):

TABLE 1

| | | |
|---|---|---|
| Macro- | Glucose | 40 |
| elements (g/l) | K₂HPO₄ | 3 |
| | Na₂HPO₄ | 3 |
| | MgSO₄•7H₂O | 0.25 |
| | (NH₄)₂SO₄ | 1 |
| | Citric acid | 1 |
| | Clerol FBA 3107 | 0.1 |
| | (antifoam) | |
| Microelements and Vitamins | CaCl₂•2H₂O | 30 |
| (mg/l) | FeSO₄•7H₂O | 1 |
| | MnSO₄•1H₂O | 8 |
| | CoSO₄•7H₂O | 0.1 |
| | CuSO₄•5H₂O | 0.2 |
| | ZnSO₄•7H₂O | 0.5 |
| | H₃BO₃ | 0.1 |
| | Na₂MoO₄•2H₂O | 0.4 |
| | Thiamine HCl | 1 |
| | Biotin | 0.015 |
| | B12 | 0.01 |
| | Calcium pantothenate | 0.03 |
| | p-Aminobenzoic acid | 0.06 |

Incubation is carried out under the following conditions: duration: 72 h; temperature: 28° C.; agitation: 110 rpm (Infors Multitron Incubator).

The preculture is then transferred to a 30 l Sartorius type fermenter.

Culture for Biomass Production:

The medium is as follows:

TABLE 2

| | | |
|---|---|---|
| Macro- | Glucose | 40 |
| elements (g/l) | KH₂PO₄ | 1.8 |
| | NaH₂PO₄ | 1.4 |
| | MgSO₄•7H₂O | 3.4 |
| | (NH₄)₂SO₄ | 0.2 |
| | Clerol FBA 3107 | 0.3 |
| | (antifoam) | |
| Microelements and | CaCl₂•2H₂O | 40 |
| Vitamins (mg/l) | FeSO₄•7H₂O | 12 |
| | MnSO₄•1H₂O | 40 |
| | CoSO₄•7H₂O | 0.1 |
| | CuSO₄•5H₂O | 0.5 |
| | ZnSO₄•7H₂O | 50 |
| | H₃BO₃ | 15 |
| | Na₂MoO₄•2H₂O | 2 |
| | Thiamine HCl | 6 |
| | Biotin | 0.1 |
| | B12 | 0.06 |
| | Calcium pantothenate | 0.2 |
| | p-Aminobenzoic acid | 0.2 |

The initial volume (Vi) of the fermenter is adjusted to 17 L after inoculation. It is brought to a final volume of approximately 20-25 l.

The parameters for performing the fermentation are as follows:

TABLE 3

| | |
|---|---|
| Temperature | 28° C. |
| pH | 5.0-5.2 by 28% w/w NH₃ |
| pO₂ | 20% ± 5% (maintained by agitation) |
| Agitation | Minimum 300 rpm |
| Air flow rate | 15 l/min |

When the residual glucose concentration falls below 10 g/l, glucose in the form of a concentrated solution at approximately 800 g/l is introduced so as to maintain the glucose content between 0 and 20 g/l in the fermenter.

Results

In 40 h, 80 g/l of biomass containing 52% of proteins are obtained.

### Example 2. Thermal Permeabilization of the Chlorella protothecoides Biomass and Recovery of the Soluble Fraction

The biomass obtained according to example 1 is:
centrifuged and washed so as to be brought to a dry matter content of 220 g/l and to a purity of more than 90% (purity defined by the ratio of the dry matter of the biomass to the total dry matter), then
thermally treated by UHT for approximately ten seconds at 135° C.

The “partially solubilized” biomass obtained in this way has of the order of 50% peptides and proteins (expressed as total amino acids), 20% sugars and 15% lipids, corresponding to a degree of solubilization of between 20% and 50% relative to the total initial biomass.

It is then separated from the soluble fraction by centrifugal separation.

The “raw” soluble substances thus contain between 60% and 75% peptides and proteins (expressed as total amino acids, of which 90% arginine and glutamic acid), between 10% and 25% sugars and less than 1% lipids.

The “depleted” biomass, in the centrifugation pellet, still has between 20% and 35% peptides and proteins (expressed as total amino acids), 25% to 35% sugars and most of all between 20% to 25% lipids.

The microfiltration permeate “P1” having a dry matter content of 5% and a titer between 60% and 75% of peptides and proteins (expressed as total amino acids) is then ultrafiltered on a membrane with a <5 kDa cut-off threshold.

The ultrafiltration retentate “R2” obtained in this way has 10% (5% to 20%) dry matter, and contains more than 90% of peptides having a molecular weight of greater than or equal to 5 kDa.

The permeate “P2” having a dry matter content of less than 3% contains peptides having a molecular weight less than 5 kDa and oligosaccharides having a DP less than or equal to 2.

This permeate “P2” can then especially be filtered on a reverse osmosis membrane (having a degree of NaCl rejection of 93%), so as to obtain:
a retentate “R3” having more than 10% dry matter, containing peptides having a molecular weight less than 5 kDa and oligosaccharides of DP 2, such as sucrose;
a permeate “R3” having 0.1% dry matter, containing oligosaccharides of DP 1, salts, free amino acids and organic acids.

The protein isolate “R2” is then:
neutralized to pH 7 with potassium hydroxide,
concentrated by evaporation to 35% dry matter (DM),
pasteurized and
atomized.

1. A method for preparing a protein isolate from the biomass of microalgae of the Chlorella genus, comprising the following steps:
providing a microalgal biomass produced by fermentation,
optionally, washing the biomass so as to eliminate the interstitial soluble compounds,
thermal permeabilization of the biomass at a temperature of between 50 and 150° C., for a duration of between 10 seconds and 5 minutes,
elimination of the biomass permeabilized in this way by a technique of solid-liquid separation chosen from the group consisting of frontal or tangential filtration, flocculation and centrifugation, more particularly multistage centrifugation, to obtain a soluble fraction,
optionally, recovery and clarification of the soluble fraction obtained in this way by microfiltration so as to remove residual insoluble substances therefrom,
ultrafiltration of the soluble fraction on a membrane with a cut-off threshold of less than 5 kDa, so as to obtain a soluble protein isolate, then
evaporation, pasteurization and atomization of said protein isolate.

2. The method as claimed in claim 1, wherein the microalgae of the Chlorella genus are chosen from the group consisting of Chlorella vulgaris, Chlorella sorokiniana and Chlorella protothecoides.

3. The method as claimed in claim 1, wherein the thermal permeabilization of the biomass is carried out at a temperature of between 100 and 150° C., for a duration of between 10 seconds and 1 minute.

4. The method as claimed in claim 1, wherein the ultrafiltration of the soluble fraction is carried out on a membrane with a cut-off threshold of between 1 and 5 kDa.

5. The method as claimed in claim 2, wherein the microalgae of the Chlorella genus is Chlorella protothecoides.
